(19) United States
(12) Patent Application Publication
(10) Pub. No.: US 2022/0401344 A1
(43) Pub. Date: Dec. 22, 2022

(54) COMPOSITIONS AND METHODS FOR TREATING KERATIN FIBERS

(71) Applicant: L'OREAL, Paris (FR)

(72) Inventors: Rachel FEREBEE MAHER, Morristown, NJ (US); Allison PERNER, Metuchen, NJ (US); Stephanie MIDORI LAGA, Rahway, NJ (US); Vibha K. SHAH, Branchburg, NJ (US)

(21) Appl. No.: 17/332,695

(22) Filed: May 27, 2021

(51) Int. Cl. A61K 8/73; A61K 8/81; A61K 8/34; A61Q 5/06

(52) U.S. Cl. CPC A61K 8/738; A61K 8/73; A61K 8/8176; A61K 8/345; A61Q 5/06

(57) ABSTRACT

The disclosure relates to compositions comprising (a) at least one cyclodextrin; (b) at least one film former; (c) at least one solvent; and (d) optionally at least one acid. The compositions can be used for imparting benefits to keratin fibers, for example temporarily straightening the hair. The disclosure also relates to methods of using the compositions.

### TECHNICAL FIELD

The present disclosure relates to compositions and methods for treating keratinous fibers, such as curly or wavy hair. The compositions provide temporary straightening properties to the hair. The disclosure also relates to methods of using the compositions.

### BACKGROUND

Consumers with curly or wavy hair often desire to temporarily straighten their hair for a change in appearance. In addition, reducing the curls of very curly hair may increase the manageability and ease of styling of such hair.

Traditional hair-straightening products generally include heavy galenics or oils, which may leave an undesirable greasy or oily feeling. Products that provide hold, such as styling gels, can also give straightening effects, but these products typically flake on hair. In addition, many hair straightening products involve heat or chemical treatments that may cause damage to the hair fibers and/or irritate the scalp.

As such, consumers desire new and improved compositions that can provide straightening effects to curly or wavy hair, and at the same time impart various additional advantageous properties to the hair such as softness, smoothness, bounce, shine, and/or frizz control.

It has now surprisingly been found that compositions comprising at least one cyclodextrin and at least one film former are able to effectively straighten curly or wavy hair without the use of heat, while at the same time delivering additional advantageous benefits to the hair.

### SUMMARY

The present disclosure relates to compositions for treating keratinous fibers, such as curly or wavy hair. The compositions may provide beneficial effects such as straightening the hair and/or providing one or more of softness, smoothness, shine, and/or frizz control to the hair. The compositions are optionally leave-in compositions.

The compositions may comprise, in certain embodiments: (a) at least one cyclodextrin or derivative thereof; (b) at least film former; and (c) at least one solvent, wherein the weight ratio of the at least one cyclodextrin or derivative thereof to the at least one film former ranges from about 1:1 to about 25:1, such as about 2:1 to about 25:1, or about 2:1 to about 20:1. In various embodiments, the at least one cyclodextrin or derivative thereof may be chosen from those of formula (I):

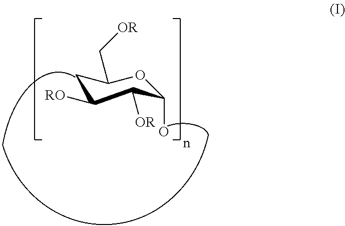

where R is chosen from H, CH₃, or a hydroxypropyl group, and n ranges from 6-8. For example, the at least one cyclodextrin or derivative thereof may be α-cyclodextrin, β-cyclodextrin, γ-cyclodextrin, methyl-α-cyclodextrin, methyl-β-cyclodextrin, methyl-γ-cyclodextrin, or a mixture of any two or more of the foregoing. In some embodiments, the at least one cyclodextrin or derivative thereof is present in an amount ranging from about 0.1% to about 15% by weight, relative to the weight of the composition, such as about 0.5% to about 10%. In various embodiments, the at least one solvent comprises water and at least one additional solvent, where the at least one additional solvent may be chosen from linear or branched, saturated or unsaturated, substituted or unsubstituted C₂-C₈ polyols. In some embodiments, the weight ratio of cyclodextrin:additional solvent ranges from about 5:1 to about 1:5, such as about 3:1 to 1:3. In various embodiments, the at least one additional solvent is chosen from hexylene glycol, pentylene glycol, or a mixture thereof. In various embodiments, the at least one film former is chosen from anionic compounds or polymers, non-ionic compounds or polymers, amphoteric compounds or polymers, zwitterionic compounds or polymers, cationic compounds or polymers, proteins, viscosity modifiers, polyacrylates, polymethacrylates, polyacrylate copolymers, polymethacrylate copolymers, polyamides, polyaminoamides, polyesters, polysaccharides, polyacrylamides, starches, gums, or mixtures thereof, such as, for example, carrageenan, xanthan gum, polyvinylpyrrolidone, or mixtures thereof. The at least one film former may, in certain embodiments, be present in an amount ranging from about 0.01% to about 5%, such as about 0.1% to about 3% by weight, relative to the total weight of the composition. Various compositions according to the disclosure may further optionally comprise at least one acid, for example at least one acid having at least one hydroxyl group, which may optionally be present in an amount ranging from about 0.01% to about 5%, such as about 0.1% to about 3% by weight, relative to the total weight of the composition. In certain embodiments, the at least one acid may be chosen from chosen from carboxylic acids and/or acids other than carboxylic acids, such as, for example, citric acid, ascorbic acid, lactic acid, malic acid, maleic acid, tartaric acid, glycolic acid, phytic acid, salts thereof, or mixtures thereof.

In other embodiments, compositions according to the disclosure may comprise: (a) from about 0.3% to about 10% of at least one cyclodextrin or derivative thereof; (b) from about 0.1% to about 3% of at least one film former; (c) at least one solvent comprising water and at least one additional solvent, wherein the at least one additional solvent is present in an amount ranging from about 0.1% to about 10%; and (d) optionally at least one acid comprising at least one hydroxyl group; wherein the weight ratio of the at least one cyclodextrin or derivative thereof to the at least one film former ranges from about 3:1 to about 25:1. In various embodiments, the at least one cyclodextrin or derivative thereof may be chosen from those of formula (I):

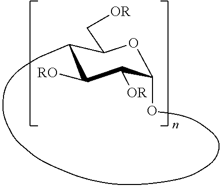

where R is chosen from H, CH₃, or a hydroxypropyl group, and n ranges from 6-8. For example, the at least one cyclodextrin or derivative thereof may be α-cyclodextrin, β-cyclodextrin, γ-cyclodextrin, methyl-α-cyclodextrin, methyl-β-cyclodextrin, methyl-γ-cyclodextrin, or a mixture of any two or more of the foregoing. In some embodiments, the at least one cyclodextrin or derivative thereof is present in an amount ranging from about 0.3% to about 8% by weight, relative to the weight of the composition, such as about 0.5% to about 5%. In various embodiments, the at least one solvent comprises water and at least one additional solvent, which may, for example, be chosen from linear or branched, saturated or unsaturated, substituted or unsubstituted C₂-C₈ polyols. In some embodiments, the weight ratio of cyclodextrin:additional solvent ranges from about 5:1 to about 1:5. In various embodiments, the at least one additional solvent is chosen from hexylene glycol, pentylene glycol, or a mixture thereof. In various embodiments, the at least one film former is chosen from anionic compounds or polymers, non-ionic compounds or polymers, amphoteric compounds or polymers, zwitterionic compounds or polymers, cationic compounds or polymers, proteins, viscosity modifiers, polyacrylates, polymethacrylates, polyacrylate copolymers, polymethacrylate copolymers, polyamides, polyaminoamides, polyesters, polysaccharides, polyacrylamides, starches, gums, or mixtures thereof, such as, for example, carrageenan, xanthan gum, polyvinylpyrrolidone, or mixtures thereof. The at least one film former may, in certain embodiments, be present in an amount ranging from about 0.1% to about 2% by weight, relative to the total weight of the composition. Exemplary compositions comprise at least one acid, for example at least one acid having at least one hydroxyl group, which may be present in an amount ranging from about 0.01% to about 5%, such as about 0.1% to about 3% by weight, relative to the total weight of the composition. In certain embodiments, the at least one acid may be chosen from chosen from carboxylic acids and/or acids other than carboxylic acids, such as, for example, citric acid, ascorbic acid, lactic acid, malic acid, maleic acid, tartaric acid, glycolic acid, phytic acid, salts thereof, or mixtures thereof.

The disclosure further relates to methods of using the compositions, for example methods of treating hair, methods of temporarily straightening hair, and/or methods of reducing frizz, wherein the methods comprise applying the composition to wet, damp, or dry hair. Optionally, the compositions are left on the hair for a period of a few hours to a few days, for example until the hair is washed. In various embodiments, the methods do not include the use of a reducing agent, and/or do not include a step of heating the hair.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a graph showing the number of passes required to break the film that had formed on each of compositions 1A-1I after drying.

FIGS. 2A-2D show images of hair swatches treated with compositions 1A-1I at T0, T0_{comb}, T1H, and T8H.

FIG. 3 is a graph demonstrating the relative change in curl for swatches treated with compositions 1A-1I from T0_{comb} to T1H to T8H.

FIGS. 4A-4D show images of hair swatches treated with compositions 2A-2I at T0, T0_{comb}, T1H, and T8H.

FIG. 5 is a graph demonstrating the relative change in curl for swatches treated with compositions 2A-2I from T0_{comb} to T1H to T8H.

FIG. 6 is a graph showing the number of passes required to break the film that had formed on each of compositions 3A-3H after drying.

FIGS. 7A-7D show images of hair swatches treated with compositions 3A-3H at T0, T0_{comb}, T1H, and T8H.

FIG. 8 is a graph demonstrating the relative change in curl for swatches treated with compositions 3A-3H from T0_{comb} to T1H to T8H.

FIG. 9 is a graph showing the number of passes required to break the film that had formed on each of compositions 4A-4F after drying.

FIGS. 10A-10D show images of hair swatches treated with compositions 4A-4F at T0, T0_{comb}, T1H, and T8H.

FIG. 11 is a graph demonstrating the relative change in curl for swatches treated with compositions 4A-4F from T0_{comb} to T1H to T8H.

It is to be understood that the foregoing and following descriptions are exemplary and explanatory only, and are not intended to be restrictive of any subject matter claimed.

### DETAILED DESCRIPTION

The disclosure relates to compositions for treating hair, as well as to methods of using the compositions.

### I. Compositions

In various embodiments, compositions according to the disclosure comprise (a) at least one cyclodextrin or derivative thereof; (b) at least one film former; (c) at least one solvent; and (d) optionally at least one acid.

Compositions according to the disclosure may provide temporary straightening effects to the hair, and in at least some embodiments may provide additional benefits such as softness, smoothness, shine, and/or frizz control to the hair.

### Cyclodextrin

Compositions according to the disclosure comprise at least one cyclodextrin, which includes derivatives thereof. Cyclodextrins are a family of cyclic oligosaccharides consisting of a macrocyclic ring of glucose subunits joined by α-1,4 glycosidic bonds.

The cyclodextrins that can be used include those of formula (I):

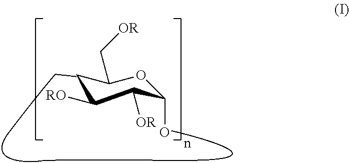

wherein:
R is chosen from H, CH₃, or a hydroxypropyl group, and
n ranges from 6-8.

For example, in embodiments where R═H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R═CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodextrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

The at least one cyclodextrin may be present in the compositions in an amount of about 0.01% to about 20% by weight, such as about 0.1% to about 18%, about 0.5% to about 15%, or about 1% to about 10% by weight, relative to the total weight of the composition. By way of non-limiting example, the at least one cyclodextrin is present in the composition with a total amount ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5% by weight, relative to the total weight of the composition, including all ranges and subranges thereof.

In one embodiment, the at least one cyclodextrin comprises or consists of β-cyclodextrin, and is present in an amount ranging from about 0.01% to about 10%, such as 0.1% to about 8%, about 0.5%% to about 7%, or about 1% to about 6% by weight, relative to the total weight of the composition, including all ranges and subranges thereof.

### Film Former

Compositions according to the disclosure comprise one or more film formers. As used herein, the terms “film former,” “film-forming agent,” “film-forming polymer,” and variations thereof, mean an agent or polymer capable, by itself or in the presence of an auxiliary film-forming agent, of forming a substantially continuous film that adheres to a support, and especially to keratin materials. In certain embodiments, the film former(s) is not crosslinked with the cyclodextrin(s).

Among the film formers that may be used, mention may be made of, for example, anionic compounds or polymers, non-ionic compounds or polymers, amphoteric compounds or polymers, zwitterionic compounds or polymers, cationic compounds or polymers, proteins, viscosity modifiers, polyacrylates, polymethacrylates, polyacrylate copolymers, polymethacrylate copolymers, polyamides, polyaminoamides, polyesters, polysaccharides, polyacrylamides, starches, gums, or mixtures of two or more of the foregoing. The film former(s) may be synthetic, semi-synthetic, natural, or plant-based.

For example, in various embodiments, the film former(s) may be chosen from anionic polymers. Without limitation, anionic film forming polymers that can be used include polymers comprising groups derived from carboxylic acid, sulfonic acid, or phosphoric acid and have a number-average molecular weight of ranging from approximately 500 and 5,000,000.

The carboxylic groups may be provided by unsaturated monocarboxylic or dicarboxylic acid monomers such as those corresponding to the formula (II):

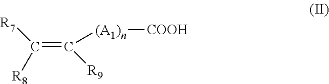

wherein n is an integer from 0 to 10, Ai denotes a methylene group optionally joined to the carbon atom of the unsaturated group or to the adjacent methylene group when n is greater than 1, via a heteroatom such as oxygen or sulfur, R7 denotes a hydrogen atom or a phenyl or benzyl group, R8 denotes a hydrogen atom or a lower alkyl or carboxyl group, and R9 denotes a hydrogen atom, a lower alkyl group, or a CH₂—COOH, phenyl or benzyl group.

By way of non-limiting example, anionic film forming polymers comprising carboxylic groups which may be used include:

(A) copolymers of acrylic or methacrylic acid or salts thereof, such as copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked, copolymers of acrylic acid and of C₁-C₄ alkyl methacrylate and terpolymers of vinylpyrrolidone, of acrylic acid and of C₁-C₂₀ alkyl methacrylate, for example lauryl methacrylate, copolymers of methacrylic acid and of ethyl acrylate, or copolymers of methacrylic acid and of methyl methacrylate; (B) crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an α- or β-cyclic carboxylic acid; and (C) copolymers of monounsaturated C₄-C₈ carboxylic acids or anhydrides including (i) copolymers comprising (a) one or more maleic, fumaric or itaconic acids or anhydrides, and (b) at least one monomer chosen from vinyl esters, vinyl ethers, vinyl halides, phenylvinyl derivatives, acrylic acid and its esters, the anhydride functions of these copolymers optionally being monoesterified or monoamidated, or (ii) copolymers comprising (a) one or more maleic, citraconic or itaconic anhydride units, and (b) one or more monomers chosen from allyl or methallyl esters optionally comprising one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

In various embodiments, the cationic film forming film-forming polymers that can be used include polymers comprising primary, secondary, tertiary and/or quaternary amine groups forming part of the polymer chain or directly attached thereto, and having a molecular weight of ranging from 500 and about 5,000,000 and preferably ranging from 1000 and 3,000,000.

For example, the cationic film-forming polymer may be chosen from: (A) homopolymers or copolymers derived from acrylic or methacrylic esters or amides, such as (i) quaternized or non-quaternized vinylpyrrolidone/dialkylaminoalkyl acrylate or methacrylate copolymers, such as the products sold under the name Gafquat® by the company ISP, for instance Gafquat® 734 or Gafquat® 755 or Gafquat® 755N (INCI name Polyquaternium-11), or alternatively the products known as Copolymer® 845, 958 and 937 sold by ISP (INCI name VP/dimethylaminoethyl methacrylate copolymer), (ii) fatty-chain polymers containing a vinylpyrrolidone unit, such as the products sold under the name Styleze® W20L and Styleze® W10 by the company ISP (INCI name Polyquaternium-55), (iii) dimethylaminoethyl methacrylate/vinylcaprolactam/vinylpyrrolidone terpolymers, such as the products sold under the names Advantage HC 37 or Gaffix® VC 713 by the company ISP (INCI name Vinyl caprolactam/VP/dimethylaminoethyl methacrylate copolymer), or (iv) quaternized vinylpyrrolidone/dimethylaminopropylmethacrylamide copolymers, such as the products sold under the name Gafquat® HS 100 by the company ISP (name Polyquaternium-28); (B) cationic guar gum derivatives, preferably containing quaternary ammonium, such as guar gums containing trialkylammonium cationic groups, for example those sold under the trade names Jaguar® C13 S, Jaguar® C 15, and Jaguar® C 17 by the company Rhodia (INCI name Guar hydroxypropyltrimonium chloride); (C) quaternary copolymers of vinylpyrrolidone and of vinylimidazole; mention may be made, for example, of vinylpyrrolidone/methylvinylimidazolium chloride copolymers, such as the products sold by the company BASF under the names Luviquat® FC550 or FC370, Luviquat® Excellence and Luviquat® Style (INCI name Polyquaternium-16), or vinylpyrrolidone/vinylimidazolium methosulfate/vinylcaprolactam terpolymers, such as the product Luviquat® Hold sold by the company BASF (INCI name Polyquaternium-46); (D) chitosans or salts thereof; the salts that can be used are, in particular, chitosan acetate, lactate, glutamate, gluconate or pyrrolidonecarboxylate, such as chitosan pyrrolidonecarboxylate sold under the name Kytamer® PC by the company Amerchol (INCI name Chitosan PCA); and (E) cationic cellulose derivatives such as copolymers of cellulose or of cellulose derivatives grafted with a water-soluble monomer comprising a quaternary ammonium, such as hydroxyalkylcelluloses, for instance hydroxymethyl-, hydroxyethyl- or hydroxypropylcelluloses grafted in particular with a methacryloyloxyethyltrimethylammonium, methacrylamidopropyltrimethyl-ammonium or dimethyldiallylammonium salt, e.g. the products sold under the name Celquat® L 200 and Celquat® H 100 by the company Akzo Nobel (INCI name Polyquaternium-4).

Exemplary amphoteric film-forming polymers that may be used in accordance with the invention may be selected from polymers comprising units B and C distributed statistically in the polymer chain, where B denotes a unit derived from a monomer comprising at least one basic nitrogen atom and C denotes a unit derived from an acid monomer comprising one or more carboxylic or sulfonic groups, or alternatively B and C may denote groups derived from carboxybetaine or sulfobetaine zwitterionic monomers, or B and C can also denote a cationic polymer chain comprising primary, secondary, tertiary or quaternary amine groups, in which at least one of the amine groups bears a carboxylic or sulfonic group connected via a hydrocarbon-based group, or alternatively B and C form part of a chain of a polymer comprising an α,β-dicarboxylic ethylene unit in which one of the carboxylic groups has been made to react with a polyamine comprising one or more primary or secondary amine groups.

For example, the amphoteric film-forming polymer may be chosen from: (A) copolymers containing acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and of a basic monomer derived from a substituted vinyl compound containing at least one basic atom, such as, more particularly, dialkylaminoalkyl methacrylate and acrylate, dialkylaminoalkylmethacrylamide and acrylamide, such as described in U.S. Pat. No. 3,836,537; (B) polymers comprising units deriving from (i) at least one monomer chosen from acrylamides or methacrylamides substituted on the nitrogen atom with an alkyl group (e.g. N-ethylacrylamide, N-tert-butylacrylamide, N-tert-octylacrylamide, N-octylacrylamide, N-decylacrylamide, N-dodecylacrylamide, or the corresponding methacrylamides), (ii) at least one acidic comonomer containing one or more reactive carboxylic groups (e.g. acrylic, methacrylic, crotonic, itaconic, maleic, or fumaric acid or alkyl monoesters of maleic or fumaric acid or anhydride having 1 to 4 carbon atoms), and (iii) at least one basic comonomer such as esters containing primary, secondary, tertiary and quaternary amine substituents of acrylic and methacrylic acids and the product of quaternization of dimethylaminoethyl methacrylate with dimethyl or diethyl sulfate (e.g. aminoethyl, butylaminoethyl, N,N′-dimethylaminoethyl or N-tert-butylaminoethyl methacrylates); (C) polymers derived from the N-carboxyalkylation of chitosan, such as N-carboxymethyl chitosan or N-carboxybutyl chitosan, for instance the product sold under the name Chitoglycan by the company Sinerga SPA (INCI name Carboxymethyl chitosan); and (D) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol.

By way of non-limiting example, non-ionic film-forming polymers may be chosen from polyalkyloxazolines; vinyl acetate homopolymers; vinyl acetate copolymers, for instance copolymers of vinyl acetate and of acrylic ester; copolymers of vinyl acetate and of ethylene, or copolymers of vinyl acetate and of maleic ester, for example of dibutyl maleate; homopolymers and copolymers of acrylic esters, for instance copolymers of alkyl acrylates and of alkyl methacrylates, such as the products provided by the company Rohm GmbH under the name Eudragit® NE 30 D (INCI name Acrylates copolymer); copolymers of acrylonitrile and of a non-ionic monomer, chosen, for example, from butadiene and alkyl (meth)acrylates; styrene homopolymers; styrene copolymers, for instance copolymers of styrene, of alkyl acrylate and of alkyl methacrylate; copolymers of styrene and of butadiene, or copolymers of styrene, of butadiene and of vinylpyridine; polyamides; vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF, polyvinylpyrrolidone (INCI name PVP); vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); and vinylpyrrolidone/methacrylamide/vinylimidazole terpolymers, for instance the product sold under the name Luviset® Clear by the company BASF (INCI name VP/methacrylamide/vinyl imidazole copolymer).

Non-limiting examples of polysaccharides that can be chosen include oxidized inulins, celluloses, starches, guar gums, xanthan gums, pullulan gums, alginate gums, agar-agar gums, carrageenan gums, gellan gums, chitosan, gums arabic, xyloses and tragacanth gums, and derivatives thereof, cellobiose, maltodextrin, scleroglucan, chitosan, ulvan, fucoidan, alginate, pectin, heparin and hyaluronic acid, or mixtures thereof. For example, gums that may be chosen include acacia, agar, algin, alginic acid, ammonium alginate, amylopectin, calcium alginate, calcium carrageenan, carrageenan, dextrin, gelatin, gellan gum, guar gum, gum Arabic, hydroxypropyl guar, guar hydroxypropyltrimonium chloride, hydroxypropyl guar hydroxypropyltrimonium chloride, karaya gum, locust bean gum, natto gum, potassium alginate, potassium carrageenan, propylene glycol alginate, sclerotium gum, sodium carboyxmethyl dextran, sodium carrageenan, tragacanth gum, xanthan gum, or any mixtures thereof.

Further non-limiting examples of polymers that can be used include non-cellulose cationic polysaccharides, such as guar gums containing trialkylammonium cationic groups. Suitable cationic guar gum derivatives include those given the PCPC (Personal Care Products Council, formerly CTFA, designation) of guar hydroxypropyl trimonium chloride, available commercially for example as JAGUAR C13S. Other suitable materials include that known as JAGUAR C15, JAGUAR C17, and JAGUAR C16 which is a hydroxypropylated cationic guar derivative containing a low level of substituent groups as well as cationic quaternary ammonium groups. Guar hydroxypropyl trimonium chloride, may also be available commercially for example as N-HANCE CG13 from the company Ashland. Also suitable is hydroxypropyl guar hydroxypropyltrimonium chloride, commercially available as JAGUAR 162.

In various embodiments, the film former(s) may be chosen from PVM/MA (Poly(methyl vinyl ether-alt-maleic acid) (e.g., sold under the name Gantrez®), carrageenan, gum Arabic, oxidized inulin, alginate, xanthan gum, xylan, chitosan, polyvinylpyrrolidone, polysilicone 8, polydimethylsiloxane, dimethylsiloxane/3-thiopropyl methyl siloxane copolymer, vinylpyyrolidone/vinylacetate copolymer, polyvinyacetate, starch, cellulose, polyquaternium-4, polyquaternium-11, acrylates/steareth-2 methacrylate crosspolymer, vinylacetate/vinyl neodecanoate copolymer, polyester-5, cetyl ethylhexanoate, vinyl acetate, crotonate/vinyl neodecanoate copolymer, 2-acryamido-2-methyl propane sulfonic acid (AMPS)/acrylic acid (AA) copolymer, AMPS/AA/acryl methacrylate copolymer, polyacrylamide, C13-C14 isoparaffin, laureth-7, octylacrylamide, acrylate/butylaminoethylmethacrylate copolymer, or any mixture thereof. In yet further embodiments, the film former may be chosen from carrageenan, xanthan gum, polyvinylpyrrolidone, or any mixture thereof. In one embodiment, the film former excludes polyvinylpyyrolidone.

The total amount of the film former present may vary, but typically ranges from about 0.001% to about 10%, such as from about 0.01% to about 5%, about 0.05% to about 3%, about 0.1% to about 2%, or about 0.25% to about 1%, based on the total weight of the composition. For example, the total amount of film former may range from about 0.01% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.25% to about 10%, about 0.25% to about 9%, about 0.25% to about 8%, about 0.25% to about 7%, about 0.25% to about 6%, about 0.25% to about 5%, about 0.25% to about 4%, about 0.25% to about 3%, about 0.25% to about 2%, about 0.25% to about 1.5%, about 0.25% to about 1%, or about 0.25% to about 0.75%, including all ranges and sub-ranges there between, based on the total weight of the composition. In certain embodiments, the at least one film forming polymer may be present in an amount of ranging from about 0.1% to about 1%, such as about 0.2% to about 0.8%, about 0.3% to about 0.7%, about 0.4% to about 0.6%, or about 0.5%, including all ranges and sub-ranges there between, based on the total weight of the composition.

In a preferred embodiment, the at least one film former comprises carrageenan, xanthan gum, polyvinylpyyrolidone, or any mixture thereof, and is present in a total amount ranging from about 0.05% to about 3%, about 0.1% to about 2%, about 0.25% to about 1%, or about 0.3% to about 0.8%, based on the total weight of the composition.

In certain embodiments, it may be desirable to choose the amount of cyclodextrin and film former to be present in a particular ratio. For example, the ratio of cyclodextrin:film former may be at least about 1:1, at least about 2:1, or at least about 3:1, and may range up to about 25:1. In various embodiments, the ratio of cyclodextrin:film former may range from about 1:1 to about 25:1, such as about 1:1 to about 20:1, about 2:1 to about 20:1, about 3:1 to about 20:1, about 4:1 to about 20:1, about 5:1 to about 20:1, about 6:1 to about 20:1, about 7:1 to about 20:1, about 8:1 to about 20:1, about 9:1 to about 20:1, or about 10:1 to about 20:1. Thus, in various embodiments, the ratio of cyclodextrin:film former may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, including all ranges using any of the aforementioned ratios as endpoints. In some embodiments, the ratio of cyclodextrin:film former may range from about 5:1 to about 25:1, about 7:1 to about 25:1, or about 10:1 to about 25:1.

In some embodiments, the at least one film former comprises carrageenan, xanthan gum, polyvinylpyyrolidone, or any mixture thereof, and the ratio of cyclodextrin:film former ranges from about 1:1 to about 25:1, such as about 1:1 to about 20:1 or about 2:1 to about 20:1. In other embodiments, the at least one film former comprises carrageenan, xanthan gum, or a mixture thereof, and the ratio of cyclodextrin:film former ranges from about 1:1 to about 25:1, such as about 1:1 to about 20:1 or about 2:1 to about 20:1. In yet further embodiments, the at least one film former comprises polyvinylpyyrolidone, and the ratio of cyclodextrin:film former ranges from about 5:1 to about 25:1, such as about 5:1 to about 20:1 or about 10:1 to about 20:1.

### Solvent

Compositions according to the disclosure comprise one or more cosmetically acceptable solvents. The solvent(s) may be chosen from water, non-aqueous solvents, or mixtures thereof. The total amount of solvent in the composition may range from about 50% to about 99% by weight, relative to the total weight of the composition, including all ranges and subranges therebetween. For example, in one embodiment, the total amount of solvent may be about 50% to about 98%, about 60% to about 98%, about 70% to about 98%, about 70% to about 95%, about 75% to 95%, or about 80% to 95% by weight, relative to the total weight of the composition.

In one embodiment, the solvent comprises water. In other embodiments, the composition comprises one or more non-aqueous solvents. For example, C₁-4 alcohols, organic solvents, fatty alcohols, fatty ethers, fatty esters, polyols, glycols, vegetable oils, mineral oils, liposomes, laminar lipid materials, or any mixture thereof. Non-limiting examples of solvents which may be used include alkane polyols such as, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-isopropyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-isopropyl ether, dipropylene glycol monomethyl ether, dipropylene glycol monoethyl ether, dipropylene glycol mono-n-propyl ether, and dipropylene glycol mono-isopropyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some exemplary embodiments, the solvent comprises water and at least one additional solvent. For example, the at least one additional solvent may be chosen from linear or branched, saturated or unsaturated, and substituted or unsubstituted polyols, for example C₂-C₈ polyols or C₃-C₆ polyols, such as diols and/or triols. Any stereoisomer of the polyols may be used.

In various exemplary embodiments, the C₂-C₈ polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, and mixtures thereof.

In one embodiment, the composition is free or substantially free of propylene glycol. In further embodiments, the composition is free or substantially free of glycols having more than two hydroxyl groups.

In a preferred embodiment, the solvent comprises water and at least one additional solvent, where the at least one additional solvent comprises, consists essentially of, or consists of hexylene glycol, pentylene glycol, or a mixture thereof.

In various embodiments, the at least one additional solvent can be present in the composition in a total amount ranging from about 0.01% to about 10%, such as from about 0.1% to about 10%, from about 0.1% to about 9%, from about 0.1% to about 8%, from about 0.1% to about 7%, from about 0.1% to about 6%, from about 0.1% to about 5%, from about 0.5% to about 10%, from about 0.5% to about 9%, from about 0.5% to about 8%, from about 0.5% to about 7%, from about 0.5% to about 6%, from about 0.5% to about 5%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 10%, from about 2% to about 9%, from about 2% to about 8%, from about 2% to about 7%, from about 2% to about 6%, or from about 2% to about 5%, by weight, relative to the total weight of the composition, including all ranges and subranges thereof.

In some embodiments, the amount of the at least one additional solvent may be chosen such that it is present in a particular weight ratio of cyclodextrin:additional solvent ranging from about 5:1 to about 1:5, such as from about 4:1 to about 1:4, from about 3:1 to about 1:3, from about 2.5:1 to about 1:2.5, or from about 2:1 to about 1:2, including all ranges and subranges thereof. In various embodiments, the ratio of cyclodextrin:additional solvent may be about 0.5:1, about 0.5:1.5, about 0.5:2, about 0.5:2.5, about 1:1, about 1:1.5, about 1:2.5, about 1.5:2, or about 1.5:2.5, including all ranges using any of the aforementioned ratios as endpoints.

In certain embodiments, the solvent is primarily comprised of water, such as from about 85% to about 99%, or about 90% to about 98%, by weight of the total solvent, with the remainder of the solvent comprising one or more additional solvents.

### Acids

Compositions according to the disclosure may optionally comprise at least one acid with at least one hydroxyl group, which includes salts thereof. The acids which may be used include, in various embodiments, mono-carboxylic acids (one carboxylic group) or poly-carboxylic acids (two or more carboxylic groups). Preferably, the carboxylic acids are non-polymeric carboxylic acids. The carboxylic acids may be chosen, in certain embodiments, from those having a molecular weight of less than about 900 g/mol, such as less than about 700 g/mol, less than about 500 g/mol, less than about 300 g/mol, or less than about 200 g/mol. Acids other than carboxylic acids may also be chosen.

In some embodiments, the at least one acid may be chosen from mono-carboxylic acids such as formic acid, acetic acid, propionic acid, butyric acid, valeric acid, caproic acid, entanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, lactic acid, or salts thereof.

In further embodiments, the at least one acid may be chosen from di-carboxylic acids such as ascorbic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, or salts thereof.

In yet further embodiments, the at least one acid may be chosen from tri-carboxylic acids such as citric acid, isocitric acid, aconitric acid, propane-1,2,3-tricarboxylic acid, benzene-1,3,5-tricarboxylic acid, or salts thereof.

In still further embodiments, the at least one acid may be chosen from acids that have at least one hydroxyl group other than carboxylic acids, such as, for example, phytic acid.

In various embodiments, the compositions comprise a mixture of two or more acids or salts thereof. For example, the compositions may comprise a mixture of two or more mono-carboxylic acids or salts thereof, or may comprise a mixture or two or more poly-carboxylic acids or salts thereof, such as two or more di-carboxylic acids, two or more tri-carboxylic acids, or a mixture of one or more di-carboxylic acids and one or more tri-carboxylic acids. Mixtures of one or more mono-carboxylic or salts thereof with one or more poly-carboxylic acids or salts thereof are also contemplated. Additionally, mixtures of carboxylic acids and acids other than carboxylic acids are also envisioned. In further embodiments, the compositions comprise only one acid or a salt thereof.

In certain embodiments, the at least one acid is chosen from citric acid, ascorbic acid, lactic acid, malic acid, maleic acid, tartaric acid, glycolic acid, phytic acid, salts thereof, or a mixture of two or more of the foregoing. In further embodiments, the at least one acid comprises, consists essentially of, or consists of citric acid, ascorbic acid, salts thereof, or a mixture thereof.

In various embodiments, the at least one acid or salt thereof may be present in the compositions in an amount of about 0.01% to about 5% by weight, such as about 0.1% to about 5%, or about 0.5% to about 5% by weight, relative to the total weight of the composition. By way of non-limiting example, the at least one carboxylic acid or salt thereof is present in the composition with a total amount ranging from about 0.01% to about 5%, by weight, for example, from about 0.01% to about 4%, about 0.01% to about 3%, about 0.01% to about 2.5%, about 0.01% to about 2%, about 0.01% to about 1.5%, about 0.01% to about 1%, about 0.1% to about 5%, about 0.1% to about 4%, about 0.1% to about 3%, about 0.1% to about 2.5%, about 0.1% to about 2%, about 0.1% to about 1.5%, about 0.1% to about 1%, about 0.5% to about 5%, about 0.5% to about 4%, about 0.5% to about 3%, about 0.5% to about 2.5%, about 0.5% to about 2%, about 0.5% to about 1.5%, or about 0.5% to about 1% by weight, relative to the total weight of the composition, including all ranges and subranges thereof. In some embodiments, the total amount of the at least one acid or salt thereof ranges from about 0.1% to about 5%, preferably from about 0.5% to about 3% by weight, relative to the total weight of the composition.

In some embodiments, the amount of the at least one acid may be chosen such that it is present in a particular weight ratio of cyclodextrin:acid ranging from about 8:1 to about 1:8, such as from about 7:1 to about 1:7, about 6:1 to about 1:6, about 5:1 to about 1:5, about 4:1 to about 1:4, from about 3:1 to about 1:3, from about 2.5:1 to about 1:2.5, or from about 2:1 to about 1:2, including all ranges and subranges thereof. In various embodiments, the ratio of cyclodextrin:additional solvent may be about 5:1, about 4:1, about 3:1, about 2:1, or about 1:1, including all ranges using any of the aforementioned ratios as endpoints.

### Additional Components

Compositions according to the disclosure may optionally comprise any additional or auxiliary component suitable for use in such compositions. Such components may include, but are not limited to, conditioning agents, dyes/pigments, moisturizing agents, fillers, structuring agents, shine agents, antioxidants, penetrants, sequestrants, fragrances, buffers, dispersants, plant extracts, preserving agents (preservatives), opacifiers, sunscreen agents, pH adjusting agents, vitamins, and antistatic agents.

Optional additional auxiliary components may be present in an amount ranging up to about 15%, such as from about 0.01% to about 15%, about 0.1% to about 10%, or about 0.1% to about 5% by weight, relative to the total weight of the composition.

In various embodiments, the compositions may have a pH less than or equal to about 7, such as about 3 to about 7, about 4 to about 7, or about 5 to about 7. In various embodiments, the pH of the compositions may range from about 2 to about 7, such as from about 3 to about 6, or from about 3.5 to about 5.5, including all ranges and subranges therebetween.

Compositions according to the disclosure are typically in the form of a liquid, cream, paste, foam, gel, or lotion, but are not limited to these forms. In some embodiments, the compositions may be in a bottle, tube, or pump-style bottle. In some embodiments, the compositions are a non-aerosol type product.

### II. Methods

The present disclosure also relates to methods for treating keratin fibers, e.g. styling hair, for example by temporarily straightening curly or wavy hair by application of the compositions described herein to the hair. The methods generally comprise applying any of the compositions according to the disclosure to the hair.

The compositions may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

In addition to temporarily straightening the hair, the compositions may also be used to provide a variety of desirable sensory benefits, for example, moisture, smoothness, softness, shine, and/or anti-frizz properties, to the hair. As such, the compositions are useful in styling (which includes shaping) hair while also caring for hair, conditioning hair, and/or imparting one or more above described sensory benefits to the hair.

Typically, methods according to the disclosure may include applying an effective amount of a composition disclosed herein to hair having waves or curls, when the hair is dry, wet, damp, or moist. The hair may be either natural or synthetic hair. As used herein, the term “effective amount” refers to an amount sufficient to provide a desired straightening effect to the hair, taking into account the degree of curliness, the length, the volume, and the texture of the hair, and/or an amount sufficient to provide a desired sensory benefit to the hair. In general, from about 0.5 grams to about 50 grams of product is applied to the hair, depending on the specific product formulation, hair length, hair volume, and hair style type. The composition applied to the hair may, for example, be distributed evenly by combing through with fingers or a means such as a comb or the like. The composition may then be dried, for example air-dried. The composition may be allowed to remain on the hair as a leave-in product for any period of time as needed, for example, a few hours or a few days, or until the next washing or rinsing of the hair. Alternatively, the compositions may be rinsed from the hair after a brief leave-in period, from about a few seconds (e.g. 5, 10, 20, 30, or 60 seconds) to about 10, 20, or 30 minutes, or longer.

In some embodiments, before applying the composition to the hair, the hair may be first cleansed with a commercially available shampoo, and/or rinsed with water, and/or dried. The composition may then be applied to the washed and/or rinsed hair when the hair is wet, damp, moist, or dry. In some other embodiments, before applying the composition to dry hair, the hair can optionally be moistened, damped, or wetted by water spray or using a wet towel, or by applying other treatment compositions that make the hair moist, damp, or wet.

In some embodiments, after application of the compositions disclosed herein, the hair may be shaped or styled as needed, such as twisted or the like. In some embodiments, the hair treated with the compositions may be twisted within 30 minutes, or within 20 minutes, while the hair is wet or damp.

Methods according to the disclosure generally do not require the use of a reducing agent, including a base, or heating the hair in order to achieve a temporary straightening effect. As such, in some embodiments, the method does not including using a reducing agent or a base, and/or does not including heating the hair.

In various embodiments, the methods of treating hair with the compositions according to the disclosure impart the benefits described above to the hair, without a greasy feel or flaking, relative to hair not having been treated with a composition according to the disclosure. The benefits imparted to the hair may remain a desired length of time, such as a few hours, a few days, or until the hair is rinsed or washed.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

Unless otherwise indicated, all percentages herein are by weight, relative to the total weight of the composition.

All percentages, parts and ratios herein are based upon the total weight of the compositions of the present disclosure, unless otherwise indicated.

As used herein, the term “treat” (and its grammatical variations, such as “treating”) refers to the application of the compositions of the present disclosure onto the surface of keratin materials, such as hair.

As used herein, the terms “applying a composition onto keratin materials,” “applying a composition onto hair,” and variations of these phrases are intended to mean contacting the keratin materials including hair and skin, with at least one of the compositions of the disclosure, in any manner. It may also mean contacting the keratin materials in an effective amount.

As used herein, the term “styling” is intended to include “shaping.”

As used herein, the term “curly hair” refers to any hair including a curl, and “wavy hair” refers to any hair including a wave. The curl and/or wave may be natural or unnatural, i.e., formed by chemical treatment or physical treatment of the hair. The degree of curliness and/or waviness of the hair may vary and is not limited.

A “leave-in” composition or product refers to a composition such as a hair-treatment composition that is not rinsed and/or washed away with water or acceptable solvent after the application of the composition onto the keratin fiber, such as hair; instead, the composition is allowed to remain on the keratin fibers for a period of time as desired, such from 1 hour, 2 hours, 3 hours, 4 hours, up to 8 hours, overnight, or as long as needed, until next time of washing or rinsing the keratin fibers.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1

The inventive compositions set forth in Table 1 below were prepared by mixing the components.

TABLE 1
Compositions

| | 1A | 1B | 1C | 1D | 1E | 1F | 1G | 1H | 1I |
|---|---|---|---|---|---|---|---|---|---|
| β-CYCLODEXTRIN | 0.5 | 5 | 10 | 0.5 | 5 | 10 | 0.5 | 5 | 10 |
| CARRAGEENAN | 0.5 | 0.5 | 0.5 | | | | | | |
| POLYVINYL- | | | | 0.5 | 0.5 | 0.5 | | | |
| PYRROLIDONE | | | | | | | | | |
| XANTHAN GUM | | | | | | | 0.5 | 0.5 | 0.5 |
| WATER | QS to | QS to | QS to | QS to | QS to | QS to | QS to | QS to | QS to |
| | 100 | 100 | 100 | 100 | 100 | 100 | 100 | 100 | 100 |

Each of compositions 1A-1I was tested on a swatch of curly hair. First, the swatches were washed and conditioned with a standard shampoo and conditioner, and excess water was squeezed from the swatches by hand. Next, each of compositions 1A-1I was applied to a swatch at a ratio of about 0.15 grams per gram of hair, and distributed through the hair by hand (10 passes) and with a comb (3 passes), after which the swatches were allowed to air dry at ambient temperature and humidity.

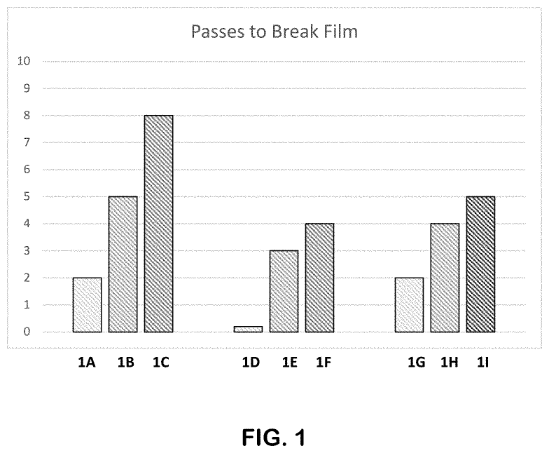

Once the swatches were dry (T0), the hair was combed with the fingers (10 passes) (T0_{comb}). FIG. 1 is a graph showing the number of passes required to break the film that had formed on each of compositions 1A-1I after drying, where a higher number of passes required to break the film correlates to a stiffer film, and a lower number of passes required to break the film correlates to a softer film. At T0_{comb}, all swatches had a smooth feel and were shiny and manageable. The swatches were then placed into a humidity chamber (80% RH) at room temperature, and evaluated after one hour (T1H) and again after eight hours (T8H).

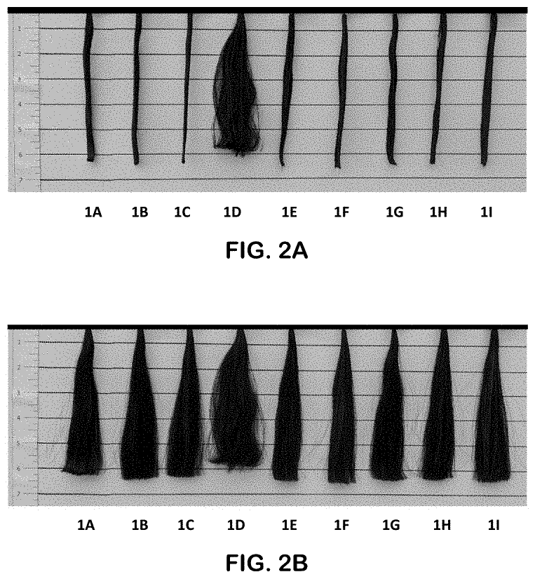

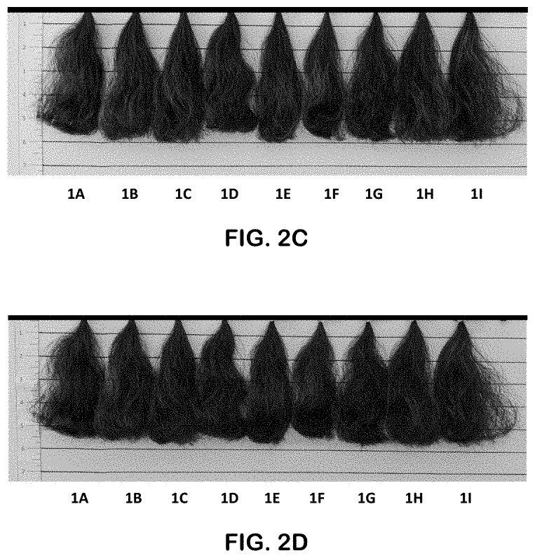

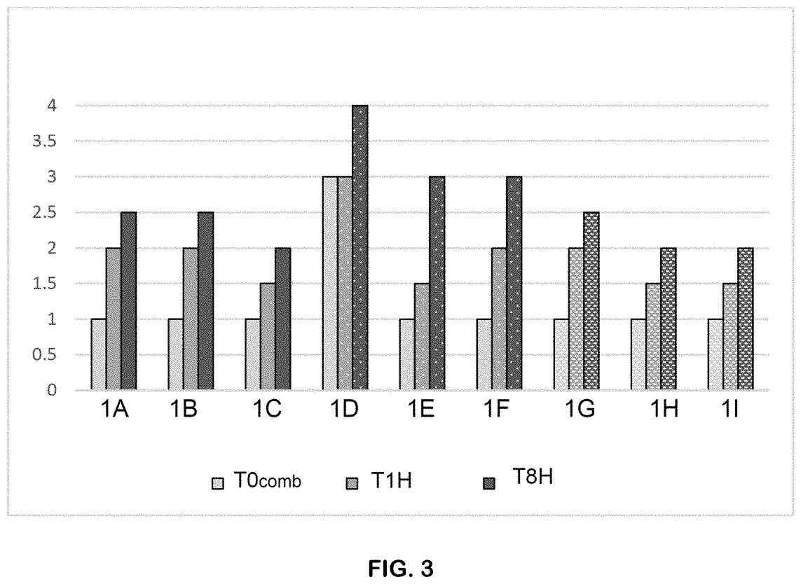

FIGS. 2A-2D are photographs of the treated swatches at T0 (FIG. 2A), T0_{comb} (FIG. 2B), T1H (FIG. 2C), and T8H (FIG. 2D). As can be seen in FIG. 2A, after drying, the treated swatches were straight other than the swatch treated with composition 1 D, which was slightly wavy. As can be seen in FIG. 2B, after combing, the swatches treated with compositions 1A-1C and 1E-1I remained straight, while the swatch treated with composition 1D, remained slightly wavy. All swatches treated with compositions 1A-1I demonstrated frizz control, and the swatches treated with compositions 1A-1C and 1E-1I were well aligned. FIGS. 2C-2D show that the swatches remained straight, even after exposure to high humidity. FIG. 3 is a graph demonstrating the relative change in curl for each swatch from T0_{comb} to T1H to T8H, where a higher value correlates to curlier hair, and a lower value correlates to straighter hair.

This Example demonstrates that hair straightening is achieved using various combinations of cyclodextrin and film former at different concentrations and ratios, even after exposure to high humidity.

### Example 2

The comparative and inventive compositions set forth in Table 2 below were prepared by mixing the components.

TABLE 2
Compositions

| | Comparative | | | | | | Inventive | | |
|---|---|---|---|---|---|---|---|---|---|
| | 2A | 2B | 2C | 2D | 2E | 2F | 2G | 2H | 2I |
| β-CYCLODEXTRIN | | 5 | | | | | 5 | 5 | 5 |
| CARRAGEENAN | | | | 0.5 | | | 0.5 | | |
| POLYVINYL- | | | | | | 0.5 | | 0.5 | |
| PYRROLIDONE | | | | | | | | | |
| XANTHAN GUM | | | | | 0.5 | | | | 0.5 |
| PENTYLENE | | | 5 | | | | 5 | 5 | 5 |
| GLYCOL | | | | | | | | | |
| WATER | QS to | QS to | QS to | QS to | QS to | QS to | QS to | QS to | QS to |
| | 100 | 100 | 100 | 100 | 100 | 100 | 100 | 100 | 100 |

Each of compositions 2A-2I was tested on a swatch of curly hair. First, the swatches were washed and conditioned with a standard shampoo and conditioner, and excess water was squeezed from the swatches by hand. Next, each of compositions 2A-2I was applied to a swatch at a ratio of about 0.15 grams per gram of hair, and distributed through the hair by hand (10 passes) and with a comb (3 passes), after which the swatches were allowed to air dry at ambient temperature and humidity.

Once the swatches were dry (T0), the hair was combed with the fingers (10 passes) (T0_{comb}), although it was noted that no swatch required more than 3 passes to break the film. At T0_{comb}, the swatches had a smooth feel. The swatches were then placed into a humidity chamber (80% RH) at room temperature, and evaluated after one hour (T1H) and again after eight hours (T8H).

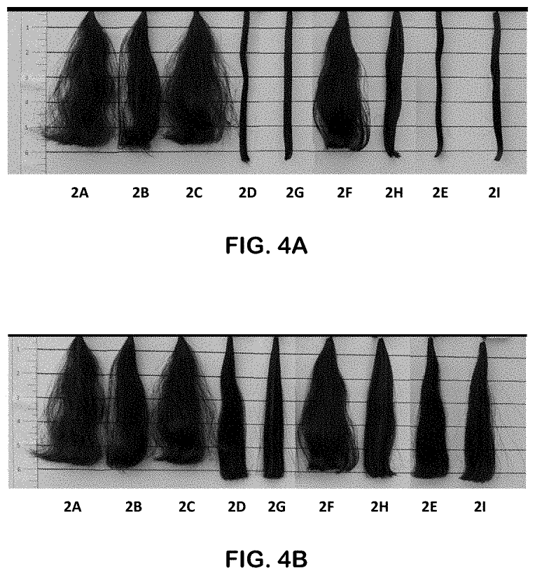

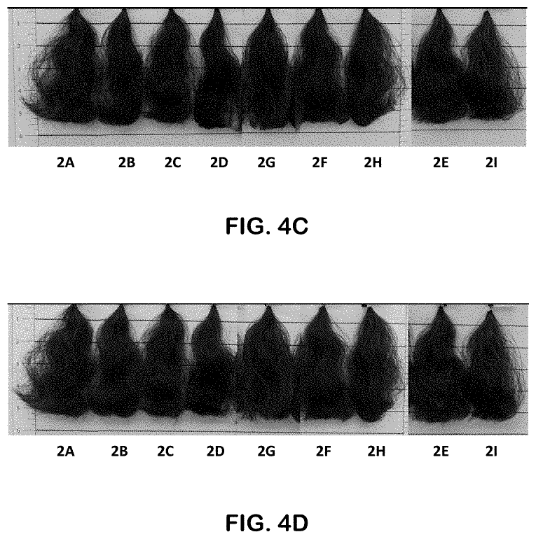

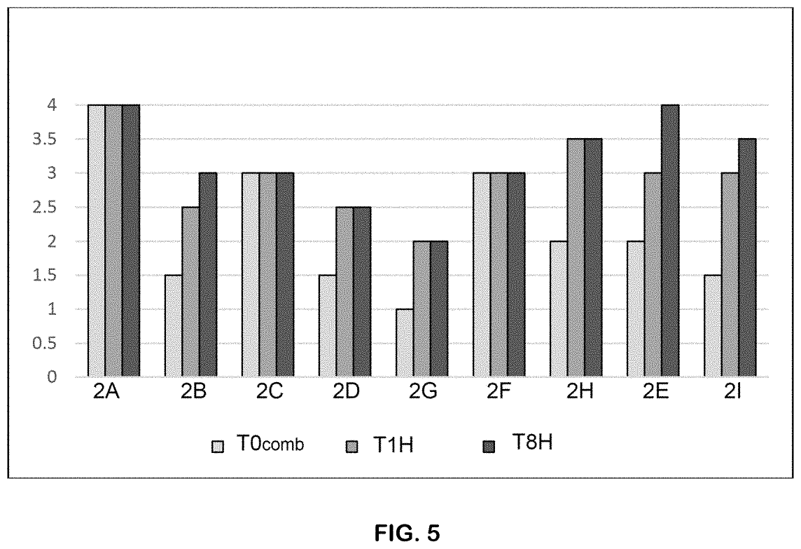

FIGS. 4A-4D are photographs of the treated swatches at T0 (FIG. 4A), T0_{comb} (FIG. 4B), T1H (FIG. 4C), and T8H (FIG. 4D). As can be seen in the figures, after drying and combing, the swatches treated with compositions 2G-2I remained the straightest, compared to the swatches treated with comparative compositions 2A-2F, with composition 2G delivering the most significant hair straightening benefits, as seen in FIG. 5. FIG. 5 is a graph demonstrating the relative change in curl for each swatch from T0_{comb} to T1H to T8H, where a higher value correlates to curlier hair, and a lower value correlates to straighter hair.

### Example 3

The comparative and inventive compositions set forth in Table 3 below were prepared by mixing the components.

TABLE 3
Compositions

| | Comparative | | | | Inventive | | | |
|---|---|---|---|---|---|---|---|---|
| | 3A | 3B | 3C | 3D | 3E | 3F | 3G | 3H |
| β-CYCLODEXTRIN | | 5 | | | 5 | 5 | 5 | 5 |
| CARRAGEENAN | | | | 0.5 | 0.5 | 0.5 | 0.5 | 0.5 |
| PENTYLENE | | | 5 | | | | 5 | 5 |
| GLYCOL | | | | | | | | |
| HEXYLENE | | | | | | 5 | | |
| GLYCOL | | | | | | | | |
| CITRIC ACID | | | | | | | | 5 |
| WATER | QS to | QS to | QS to | QS to | QS to | QS to | QS to | QS to |
| | 100 | 100 | 100 | 100 | 100 | 100 | 100 | 100 |

Each of compositions 3A-3H was tested on a swatch of curly hair. First, the swatches were washed and conditioned with a standard shampoo and conditioner, and excess water was squeezed from the swatches by hand. Next, each of compositions 3A-3H was applied to a swatch at a ratio of about 0.15 grams per gram of hair, and distributed through the hair by hand (10 passes) and with a comb (3 passes), after which the swatches were allowed to air dry at ambient temperature and humidity.

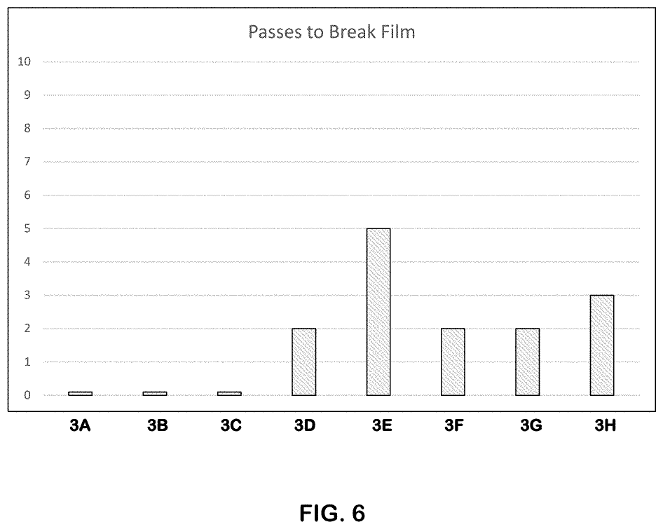

Once the swatches were dry (T0), the hair was combed with the fingers (10 passes) (T0_{comb}). FIG. 6 is a graph showing the number of passes required to break the film that had formed on each of compositions 3A-3H after drying, where a higher number of passes required to break the film correlates to a stiffer film, and a lower number of passes required to break the film correlates to a softer film. At T0_{comb}, all swatches had a smooth feel and were shiny and manageable. The swatches were then placed into a humidity chamber (80% RH) at room temperature, and evaluated after one hour (T1H) and again after eight hours (T8H). The swatches treated with compositions 3F and 3G felt the smoothest.

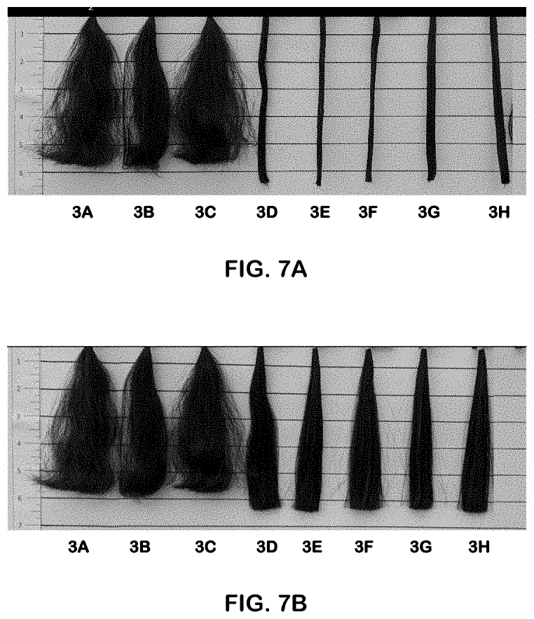

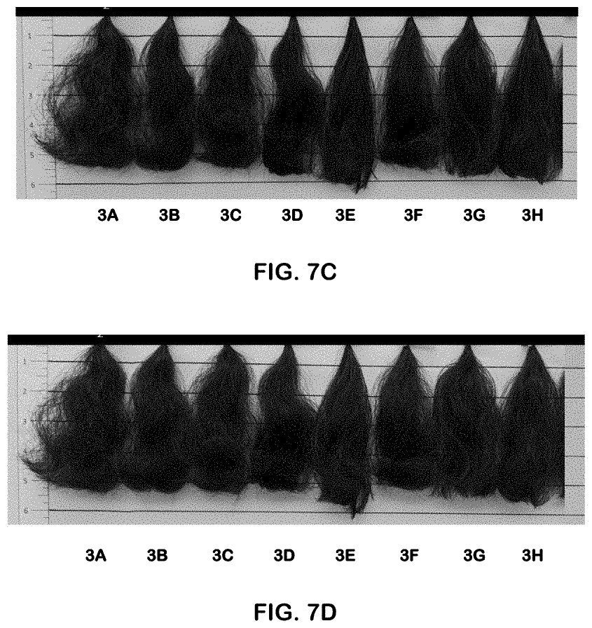

FIGS. 7A-7D are photographs of the treated swatches at T0 (FIG. 7A), T0_{comb} (FIG. 7B), T1H (FIG. 7C), and T8H (FIG. 7D). As can be seen in FIG. 7B, after the film was broken (T0_{comb}), the swatches treated with inventive compositions 3E-3H were straighter than the swatches treated with compositions 3A-3D. As seen in FIGS. 7C-7D, the hair treated with inventive compositions 3E-3H remained straighter and had less frizz, even after exposure to high humidity, than the swatches treated with compositions 3A-3D.

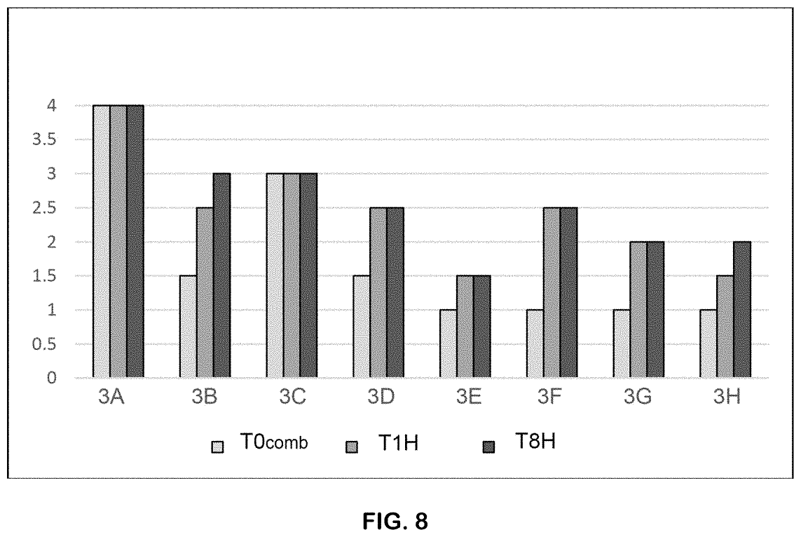

The increase in straightening benefit to hair using compositions 3E-3H is confirmed in FIG. 8, which is a graph demonstrating the relative change in curl for each swatch from T0_{comb} to T1H to T8H, where a higher value correlates to curlier hair, and a lower value correlates to straighter hair. FIG. 8 shows the least curl (i.e. most straightening) at T0_{comb}, T1H, and T8H for swatches treated with compositions 3E-3H.

This Example demonstrates that compositions according to the disclosure work to synergistically provide straightening benefits which persist over time and at high humidity, and also provide sensorial benefits to the hair, which benefits were unexpected and surprising compared to the treatment by the individual components alone.

### Example 4

The inventive compositions set forth in Table 4 below were prepared by mixing the components.

TABLE 4
Compositions

| | 4A | 4B | 4C | 4D | 4E | 4F |
|---|---|---|---|---|---|---|
| β-CYCLODEXTRIN | 5 | 5 | 5 | 5 | 5 | 5 |
| CARRAGEENAN | 0.5 | 0.5 | 0.5 | 0.5 | 0.5 | 0.5 |
| PENTYLENE | 2.5 | 5 | 10 | 5 | 5 | 5 |
| GLYCOL | | | | | | |
| CITRIC ACID | | | | 5 | 2.5 | 1 |
| WATER | QS to | QS to | QS to | QS to | QS to | QS to |
| | 100 | 100 | 100 | 100 | 100 | 100 |

Each of compositions 4A-4F was tested on a swatch of curly hair. First, the swatches were washed and conditioned with a standard shampoo and conditioner, and excess water was squeezed from the swatches by hand. Next, each of compositions 4A-4F was applied to a swatch at a ratio of about 0.15 grams per gram of hair, and distributed through the hair by hand (10 passes) and with a comb (3 passes), after which the swatches were allowed to air dry at ambient temperature and humidity.

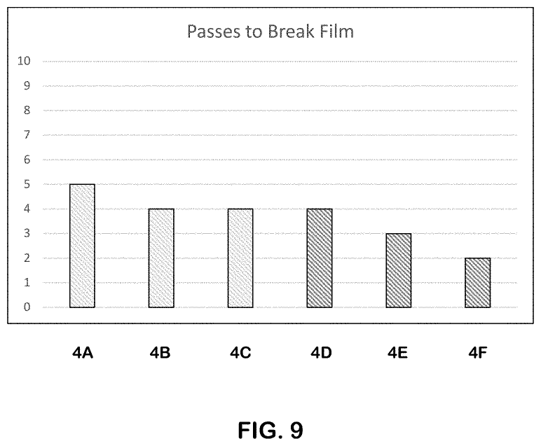

Once the swatches were dry (T0), the hair was combed with the fingers (10 passes) (T0_{comb}). FIG. 9 is a graph showing the number of passes required to break the film that had formed on each of compositions 4A-4F after drying, where a higher number of passes required to break the film correlates to a stiffer film, and a lower number of passes required to break the film correlates to a softer film. At T0_{comb}, all swatches had a smooth feel and were shiny and manageable. The swatches were then placed into a humidity chamber (80% RH) at room temperature, and evaluated after one hour (T1H) and again after eight hours (T8H).

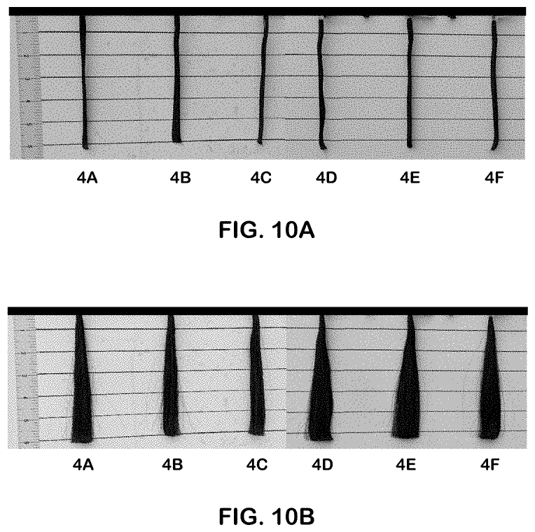

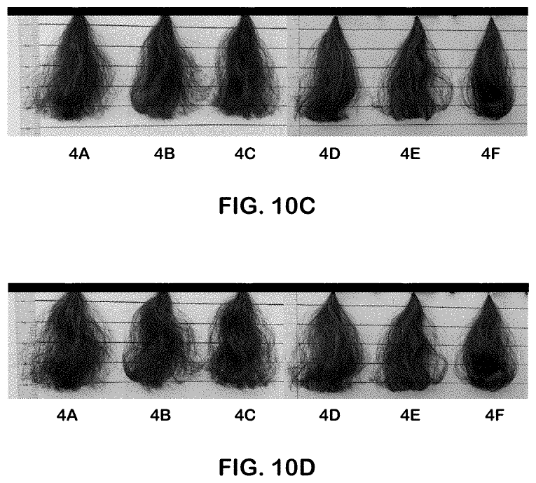

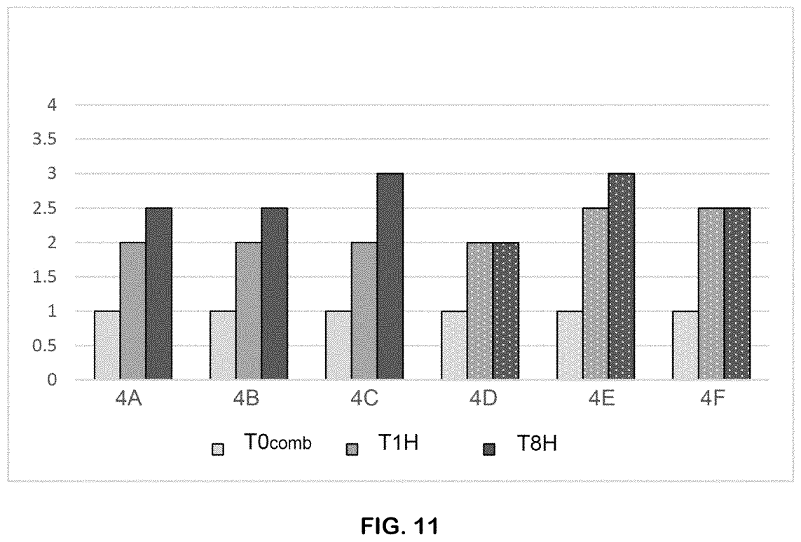

FIGS. 10A-10D are photographs of the treated swatches at T0 (FIG. 10A), T0_{comb} (FIG. 10B), T1H (FIG. 10C), and T8H (FIG. 10D). As can be seen in FIGS. 10A-10B, after drying and combing, the treated swatches were straight and aligned. All swatches treated with compositions 4A-4F demonstrated frizz control. FIGS. 10C-10D show that the swatches remained straight, even after exposure to high humidity. FIG. 11 is a graph demonstrating the relative change in curl for each swatch from T0_{comb} to T1H to T8H, where a higher value correlates to curlier hair, and a lower value correlates to straighter hair. FIG. 11 shows that all of compositions 4A-4F provide straightening benefits that persist even after exposure to high humidity.

This Example demonstrates that effective hair straightening is achieved using various combinations of cyclodextrin, film former, solvent including a polyol, and acid at different concentrations and ratios.

### Example 5

The inventive compositions set forth in Table 5 below were prepared by mixing the components.

TABLE 5
Compositions

| | 5A | 5B | 5C | 5D |
|---|---|---|---|---|
| β-CYCLODEXTRIN | 4.3 | 4.3 | 4.3 | 4.3 |
| CARRAGEENAN | 0.5 | 0.5 | | |
| POLYVINYLPYRROLIDONE | | | 0.5 | |
| XANTHAN GUM | | | | 0.5 |
| PENTYLENE GLYCOL | 5 | 5 | 5 | 5 |
| CITRIC ACID | | 1 | | |
| WATER | QS | QS | QS | QS |
| | to 100 | to 100 | to 100 | to 100 |

The above Examples demonstrate that compositions according to the disclosure provide hair straightening benefits and/or sensory benefits such as smoothness, shine, and/or frizz control to hair, which lasts over time even after exposure to high humidity, over a range of different ratios, concentrations, and components.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A composition for treating keratin fibers, comprising:
(a) at least one cyclodextrin or derivative thereof;
(b) at least one film former; and
(c) at least one solvent;
wherein the weight ratio of the at least one cyclodextrin or derivative thereof to the at least one film former ranges from about 2:1 to about 25:1.

2. The composition of claim 1, wherein the at least one cyclodextrin or derivative thereof is chosen from those of formula (I): 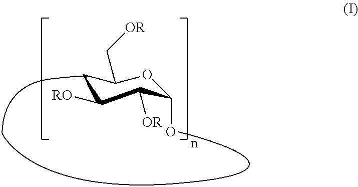
wherein:
R is chosen from H, CH₃, or a hydroxypropyl group, and
n ranges from 6-8.

3. The composition of claim 1, wherein the at least one cyclodextrin or derivative thereof is chosen from α-cyclodextrin, β-cyclodextrin, γ-cyclodextrin, methyl-α-cyclodextrin, methyl-β-cyclodextrin, methyl-γ-cyclodextrin, or mixtures thereof.

4. The composition of claim 1, wherein the at least one cyclodextrin or derivative thereof is present in an amount ranging from about 0.5% to about 15% by weight, relative to the weight of the composition.

5. The composition of claim 1, wherein the at least one solvent comprises water and at least one additional solvent.

6. The composition of claim 5, wherein the at least one additional solvent is chosen from linear or branched, saturated or unsaturated, substituted or unsubstituted C₂-C₈ polyols.

7. The composition of claim 5, wherein the weight ratio of cyclodextrin:additional solvent ranges from about 5:1 to about 1:5.

8. The composition of claim 7, wherein the at least one additional solvent is chosen from hexylene glycol, pentylene glycol, or a mixture thereof.

9. The composition of claim 1, wherein the at least one film former is chosen from anionic compounds or polymers, non-ionic compounds or polymers, amphoteric compounds or polymers, zwitterionic compounds or polymers, cationic compounds or polymers, proteins, viscosity modifiers, polyacrylates, polymethacrylates, polyacrylate copolymers, polymethacrylate copolymers, polyamides, polyaminoamides, polyesters, polysaccharides, polyacrylamides, starches, gums, or mixtures thereof.

10. The composition of claim 9, wherein the at least one film former is chosen from carrageenan, xanthan gum, polyvinylpyrrolidone, or mixtures thereof.

11. The composition of claim 1, wherein the at least one film former is present in an amount ranging from about 0.01% to about 5% by weight, relative to the total weight of the composition.

12. The composition of claim 1, wherein the wherein the weight ratio of cyclodextrin or derivative thereof to the at least one film former ranges from about 2:1 to about 20:1.

13. The composition of claim 1, further comprising at least one acid comprising at least one hydroxyl group.

14. The composition of claim 13, wherein the at least one acid comprising at least one hydroxyl group is present in an amount ranging from about 0.1% to about 5% by weight, relative to the total weight of the composition.

15. The composition of claim 13, wherein the at least one acid is chosen from citric acid, ascorbic acid, lactic acid, malic acid, maleic acid, tartaric acid, glycolic acid, phytic acid, salts thereof, or mixtures thereof.

16. A composition for treating keratin fibers, comprising:
(a) from about 0.3% to about 10% of at least one cyclodextrin or derivative thereof;
(b) from about 0.1% to about 3% of at least one film former;
(c) at least one solvent comprising water and at least one additional solvent, wherein the at least one additional solvent is present in an amount ranging from about 0.1% to about 10%; and
(d) optionally at least one acid comprising at least one hydroxyl group;
wherein the weight ratio of the at least one cyclodextrin or derivative thereof to the at least one film former ranges from about 3:1 to about 25:1.

17. The composition of claim 16, wherein the at least one cyclodextrin or derivative thereof is chosen from α-cyclodextrin, β-cyclodextrin, γ-cyclodextrin, methyl-α-cyclodextrin, methyl-β-cyclodextrin, methyl-γ-cyclodextrin, or mixtures thereof.

18. The composition of claim 16, wherein the at least one additional solvent is chosen from linear or branched, saturated or unsaturated, substituted or unsubstituted C₂-C₈ polyols.

19. A method for treating hair comprising applying to the hair a composition according to claim 16.

20. A method for treating hair comprising applying to the hair a composition comprising:
(a) at least one cyclodextrin or derivative thereof;
(b) at least one film former; and
(c) at least one solvent;
wherein the weight ratio of the at least one cyclodextrin or derivative thereof to the at least one film former ranges from about 2:1 to about 25:1.
